(19) United States
(12) Patent Application Publication
(10) Pub. No.: US 2024/0090807 A1
(43) Pub. Date: Mar. 21, 2024

(54) WEARABLE DEVICE AND METHOD FOR STRESS DETECTION, EMOTION RECOGNITION AND EMOTION MANAGEMENT

(71) Applicant: STRESSLESS SRL, Cluj Napoca (RO)

(72) Inventors: Marius Dan RUS, Cluj (RO); Adina Viorica RUS, Cluj (RO); Florin Codrut LAZAR, Cluj (RO); Victor Paul BARNA, Bistrita Nasaud (RO)

(21) Appl. No.: 18/038,417

(22) PCT Filed: Dec. 30, 2021
(86) PCT No.: PCT/IB2021/062448

§ 371 Date: May 23, 2023

Related U.S. Application Data
(60) Provisional application No. 63/131,933, filed on Dec. 30, 2020.

(51) Int. Cl. A61B 5/16; A61B 5/00; A61B 5/0205

(52) U.S. Cl. CPC A61B 5/165; A61B 5/02055; A61B 5/486; A61B 5/7246; A61B 5/726; A61B 5/7267; A61B 5/7278; A61B 5/7282; A61B 5/742; A61B 5/746; A61B 5/0533

(57) ABSTRACT

A wearable device is configured to perform stress detection, emotion recognition and emotional management. The wearable device may perform a process that includes monitoring an emotional score of a user, the emotional score computed based on measured biological signals of the user. The device may detect an emotional event from the monitored emotional score. The device may transmit an alert signal to the user in response to the detected emotional event, in which an action plan associates the alert signal with a corresponding action to be performed by the user. The device may evaluate an effectiveness of the action plan by evaluating an impact of the action on the emotional score of the user. The device may determine a change to the action plan based on the evaluated effectiveness of the action plan. Finally, the device may incorporate the determined change into the action plan.

### RELATED APPLICATIONS

This application is a National Stage under 35 USC 371 of and claims priority to International Application No. PCT/IB2021/062448, filed 30 Dec. 2021, which claims the priority benefit of U.S. Provisional Application No. 63/131,933, filed 30 Dec. 2020.

### FIELD OF THE INVENTION

The present invention relates to the field of wearable electronic devices, and more specifically relates to a device and method for stress identification, emotion recognition and management.

### BACKGROUND

Coping with stress and recognizing and controlling one's negative emotions is something everybody is doing on a daily basis, but for some people this is harder than for others. This is a particularly pressing issue for people affected by certain mental health related conditions, such as autism, attention deficit and hyperactivity disorder (ADHD), post-traumatic stress disorder (PTSD) or bipolar disorder, where emotional manifestations are externalized, but also for people affected with other conditions, such as anxiety and panic attacks, where the emotional manifestations are internalized.

These are conditions that are often marked by ongoing patterns of hyperactivity and/or impulsivity, triggered by daily stress, and interfere with social functioning, at school or in the workplace, and with the person's overall development. The most common symptom of externalizing manifestations conditions are connected with hyperactivity and impulsivity, which mean that the affected person seems to move about constantly, including in situations in which it is not appropriate. Occasionally, these situations may include the person exhibiting emotional flares, in which the affected person makes hasty actions, including violent actions with a high potential for harm, without initial consideration.

Current solutions to such conditions include medication, which may have significant side effects and age limitations (not advisable below the ages of 7-10, depending on the active compound), and psychological therapy, which may provide gradual benefits only on a long term horizon, and may have limited capability for immediate and direct behavioral influence during social interactions, especially for children, who manifest the lowest amount of self-control, for whom specialized counseling may be necessary. For example, trained observers may use simple attention distraction or meditation to calm down children before the onset of the emotional flare.

Another solution for children and teenagers, which greatly improves the efficiency of therapy, is classroom intervention, usually provided in schools. Classroom intervention may help the children identify the stressors and deal with them at the exact moment of need during classes, but comes with high costs and low penetration as it requires trained personnel and one-to-one interaction with the individuals. However, classroom intervention is far from being as widely available as needed. A Lehigh University study focused on ADHD (DuPaul, 2019) published in March 2019, found that out of 2,495 children with ADHD, one in three received no school-based interventions and two out of five received no classroom management. At least one in five students who experience significant academic and social impairment, those most in need of services, received no school intervention whatsoever.

Until this date, the academic research has been mostly focused on studying the effects and correlation of stress with Galvanic Skin Response (skin conductivity) and Heart Rate. Some example papers describing this aspect include:
Nurdina Widanti, Budi Sumanto, Poppy Rosa, M. Fathur Miftahudin, “Stress level detection using heart rate blood pressure and GSR and stress therapy by utilizing infrared”, International Conference on Industrial Instrumentation and Control (ICIC) 2015, pp. 275-279, 2015.
Monika Chauhan, Shivani V. Vora, Dipak Dabhi, “Effective stress detection using physiological parameters”, International Conference on Innovations in Information Embedded and Communication Systems (ICIIECS) 2017, pp. 1-6, 2017.
Atlee Fernandes, Rakesh Helawar, R. Lokesh, Tushar Tari, Ashwini V. Shahapurkar, “Determination of stress using Blood Pressure and Galvanic Skin Response”, International Conference on Communication and Network Technologies (ICCNT) 2014, pp. 165-168, 2014.

It is a logical conclusion that modern technology, especially sensor technology, could be used for better solutions; however, there have been limited inventions focused on this field. Currently, there is only one type of device used for alleviating the effects of stress and anxiety through the use of random patterns of vibrations that counter external stressors, but without any personalization on the patients' specific condition and mental state. There are also several wearable devices working on emotion tracking, such as emotional sensors developed by mPath™ of Broomfield CO, the Upmood watch developed by Upmood Design Lab™ of San Francisco, CA, and the Feel Emotion Sensor developed by Sentio Solutions' of San Francisco, CA. The amount of data collected and the types of sensors used differ, and so do the use cases.

Described herein are techniques that improve upon the prior techniques and devices for stress detection, emotion recognition and emotion management.

### SUMMARY OF THE INVENTION

Concepts discussed herein relate to a wearable device or apparatus for monitoring biometric data of a user and enabling biofeedback indications in response to biometric data received in order to serve as an early warning system for the user and/or to guide the user through coping with the identified stress or emotions. In one particular embodiment, the wearable device includes at least some of a set of sensors (e.g., used for measuring and calculating parameters including heart rate, heart rate variability, blood oxygenation, galvanic skin response (GSR), skin temperature, pulse rate (also called heart rate), blood pressure, position and movement), a button, at least one digital signal processor having a memory unit coupled to at least one of the sensors and a feedback mechanism. The feedback mechanism can include a vibration actuator and/or a set of light emitting diodes. Further, the memory stores computer-executable instructions for controlling the at least one processor to cause the sensor to collect data continuously (or in response to the activation signal from the button) and to process the collected data. Furthermore, the concepts include the algorithm used for detecting the emergence of an emotional event and for launching a warning and/or an intervention process. Furthermore, the device is configured to provide user feedback, through a feedback mechanism, with reference to the collected data. One embodiment of the feedback mechanism further comprises a vibration actuator as a biofeedback indicator and the feedback to the user is provided as haptic vibration. Another embodiment of the feedback mechanism comprises a set of LEDs and the feedback is provided to the user as patterns of light.

These and other embodiments of the invention are more fully described in association with the drawings below.

### BRIEF DESCRIPTION OF THE DRAWINGS

FIG. 1 depicts an exploded view of the wearable device, according to one embodiment of the present invention.

FIG. 2 illustrates a bottom and top view of the PCB, according to one embodiment of the present invention.

FIG. 3 illustrates process that may occur when the wearable device 10 is worn by a user, according to one embodiment of the present invention.

FIG. 4A illustrates a flow diagram of a detection and intervention algorithm, according to one embodiment of the present invention.

FIG. 4B illustrates a flow diagram of another detection and intervention algorithm, according to one embodiment of the present invention.

FIG. 5 depicts the emotional score and an agitation level (over time) of a user during the same period of time, according to one embodiment of the present invention.

FIGS. 6A and 6B depict the respective emotional scores for two users over time. The data from a user diagnosed with ADHD is depicted in FIG. 6A, and the data from a user that was not diagnosed with any mental health conditions is depicted in FIG. 6B.

FIG. 7 depicts several possibilities of wearing the device on a user, according to one embodiment of the present invention.

FIG. 8 depicts an illustration of components of the wearable device, according to one embodiment of the present invention.

FIG. 9 depicts a hardware and software architecture of the wearable device, according to one embodiment of the present invention.

FIG. 10 depicts a flow diagram of a process for processing the photoplethysmography (PPG) signals collected by one or more sensors, according to one embodiment of the present invention.

FIG. 11 depicts a flow diagram that provides an overview of a process for generating biofeedback to the one or more features extracted from the measured signals, according to one embodiment of the present invention.

FIG. 12 depicts a flow diagram of a process for discriminating between samples that should be stored and processed and samples that should be discarded, according to one embodiment of the present invention.

FIG. 13 depicts a flow diagram of a process to extract features from certain frequency bands from the PPG signals, according to one embodiment of the present invention.

FIG. 14 depicts a flow diagram of a process for training and initializing the machine learning (ML) model for inferring stressful events from the sensed signals, according to one embodiment of the present invention.

FIG. 15 depicts a flow diagram of a process for updating the ML model based on feedback from the user received from the physical button on the wearable device, according to one embodiment of the present invention.

FIG. 16 depicts a flow diagram of a process for updating the ML model using supervised learning, according to one embodiment of the present invention.

FIG. 17 depicts a flow diagram of a method of using the alert signals from the high intensity emotional events forecasting process to manage the behavioral response of the user to emotional events, to personalize the actions for the user to perform in response to emotional events and to teach the user the appropriate responses to emotional events, according to one embodiment of the present invention.

FIG. 18 depicts components of a computer system in which computer readable instructions instantiating the methods of the present invention may be stored and executed.

### DETAILED DESCRIPTION

Various embodiments and aspects of the inventions will be described with reference to details discussed below, and the accompanying drawings will illustrate the various embodiments. The following description and drawings are illustrative of the invention and are not to be construed as limiting the invention. Numerous specific details are described to provide a thorough understanding of various embodiments of the present invention. However, in certain instances, well-known or conventional details are not described in order to provide a concise discussion of embodiments of the present inventions. Reference in the specification to “one embodiment” or “an embodiment” or “another embodiment” means that a particular feature, structure, or characteristic described in conjunction with the embodiment can be included in at least one embodiment of the invention. The appearances of the phrase “in one embodiment” in various places in the specification do not necessarily all refer to the same embodiment.

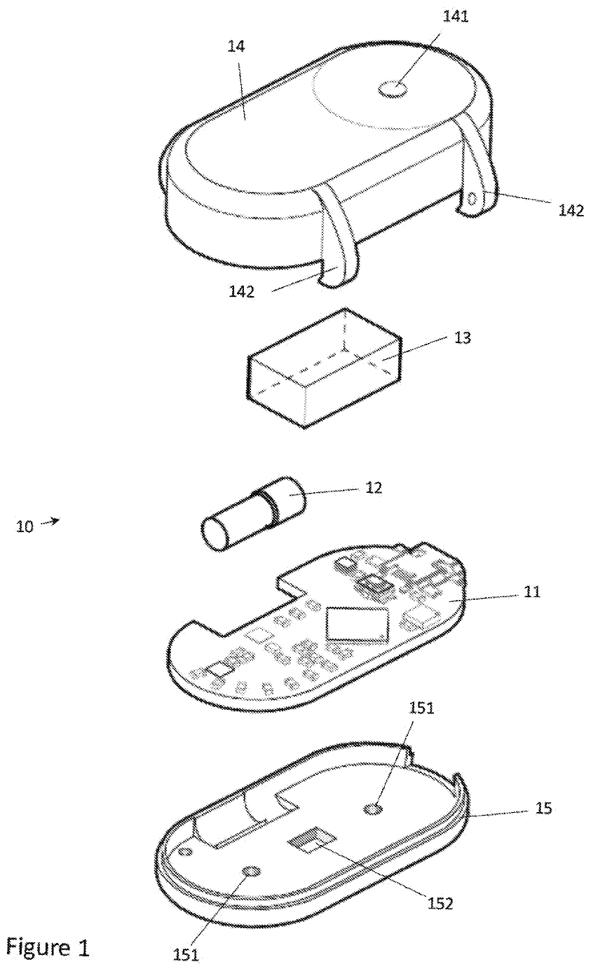

FIG. 1 is an exploded view of a wearable electronic device 10 for stress detection, emotion recognition, and emotion management. The wearable electronic device 10 may include a printed circuit board (PCB) 11 that contains the processor and a plurality of skin sensors configured to measure one or more biomarkers of a user via contact or proximity to the skin of the user. In the embodiment illustrated in FIG. 1, the wearable electronic device 10 may further include a motor 12 for providing vibration feedback, and a battery 13. The wearable electronic device 10 may also include an upper case 14 and a bottom case 15. The upper case 14 may include a button 141 and one or more arms 142 for one or more straps that fix the device on the user. In at least one embodiment, the arms 142 are configured to allow the wearable electronic device 10 to be worn around a wrist of a user. The bottom case 15 may include openings 151 and 152 to allow the skin conductivity sensor and optical sensors to connect or come into proximity of the user's skin so as to collect biomarkers. In one or more embodiments, one or more of the upper case 14, bottom case 15 and PCB 11 may be composed of a flexible material, allowing bending of the wearable electronic device 10 around the wrist or other body part of the user.

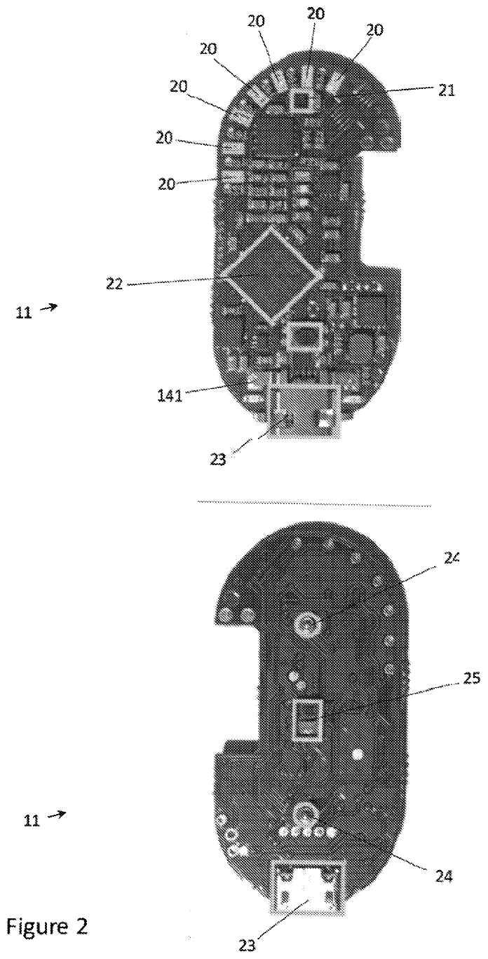

FIG. 2 illustrates a top view (top portion) and bottom view (bottom portion) of the PCB 11. As shown in FIG. 2, the PCB 11 may include a control module 22 which in one embodiment is represented by a microcontroller unit. The control module 22 may include components, circuitry, and/or logic configured to receive the measured one or more biomarkers, determine a stress and/or or emotional state of the user based upon the received parameters, and provide feedback to the user via haptic vibration and/or LED patterns. The PCB 11 may include a button 141 to allow the user to start and stop of the monitoring session, and a connection 23 to receive power and to receive or transmit data. The PCB 11 may include an accelerometer 21 for collecting movement related information. The PCB 11 may include one or more light emitting diodes (LED) 20 serving as feedback delivery mechanism, through the generation of light patterns. The PCB 11 may include a plurality of sensors 25 for collecting biomarkers of the user, including but not limited to, skin temperature sensors, and optical sensors on multiple wavelengths for heart rate and oxygen concentration monitoring. Furthermore, the PCB 11 may include electrodes 24 for measuring galvanic skin resistance and/or electrocardiogram (ECG) sensors (not depicted) for monitoring heart activity.

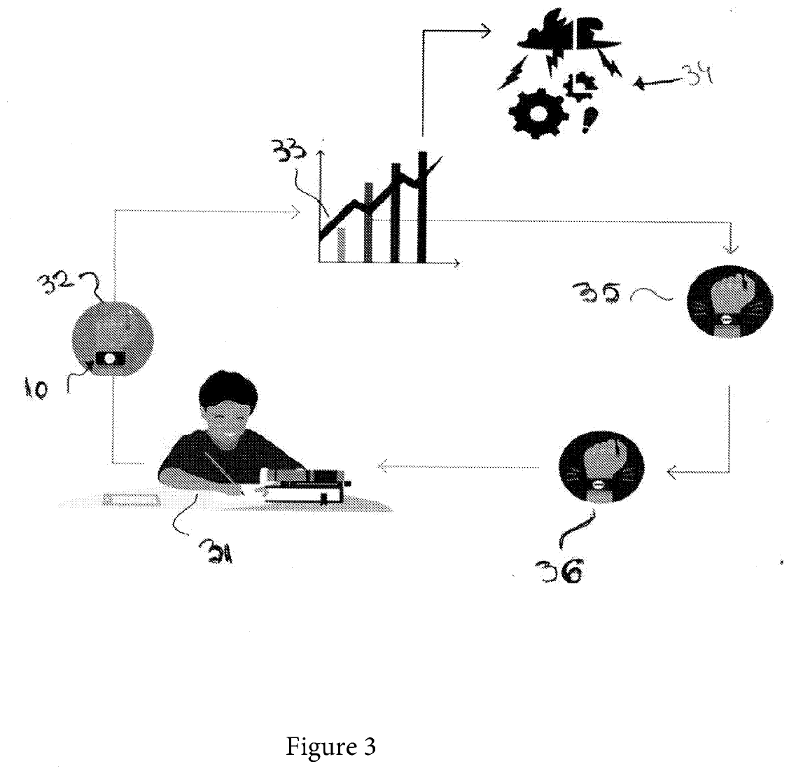

FIG. 3 illustrates a process that may occur when the wearable device 10 is worn by a user. In the example, the process illustrates events that may occur when a child who has been diagnosed with attention deficit hyperactivity disorder (ADHD) wears a wrist based embodiment of the device 10. It is, however, understood that users whether young or old and/or whether with or without mental conditions may utilize the device 10.

In one embodiment, the process begins with the user in a calm emotional state, and the user wearing the device 10 around his/her wrist (step 31). The device 10 constantly monitors (step 32) a plurality of biomarkers and based on them, evaluates (step 33) the user's emotional score, and compares it with a baseline derived threshold for intervention. The mental stress and intellectual challenge from the current activity gradually affects the user and increase his/her overall stress to levels where, without an intervention, an emotional event (step 34) would manifest, in which the user could lose partial or complete control of their behavior and perform potentially harmful and/or violent actions. The wearable device 10 detects this emotional escalation and launches an alert signal (step 35) through the haptic vibration and/or light pattern feedback mechanism of the device 10, alerting the user about his/her emotional state and starting the intervention process (step 36). The device 10 continues the biofeedback based intervention until the user returns to a calm emotional state (step 31) and the process continues in a similar manner as described above until the session is terminated by the user.

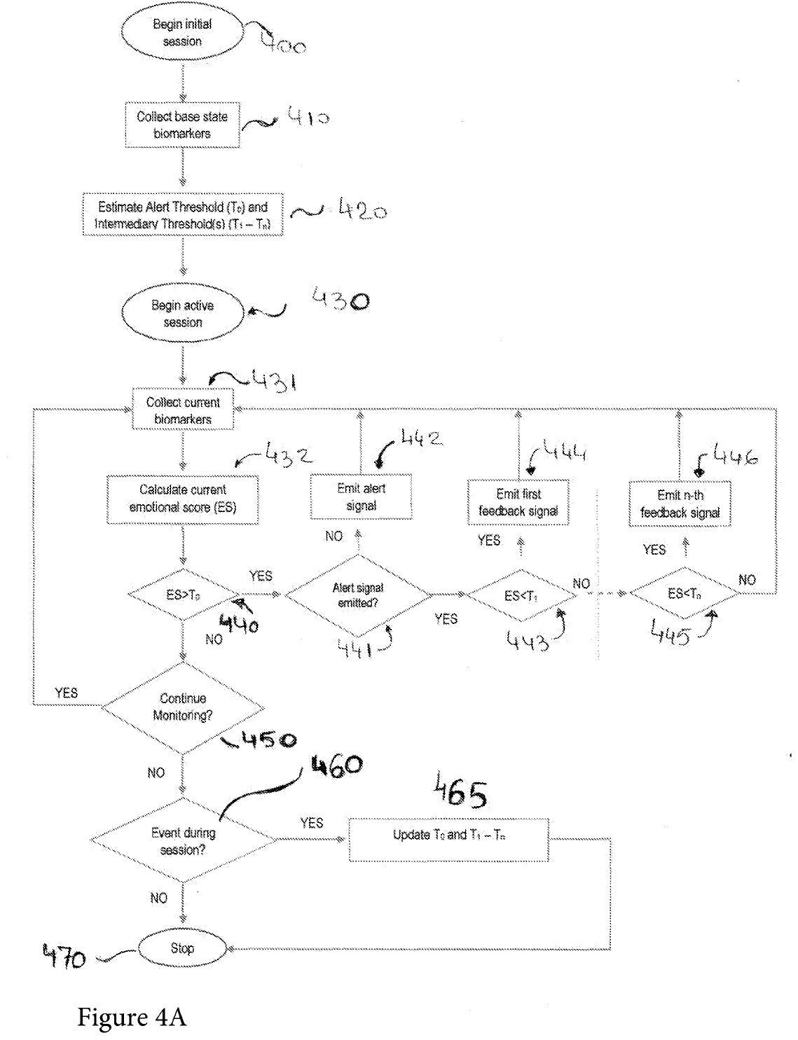

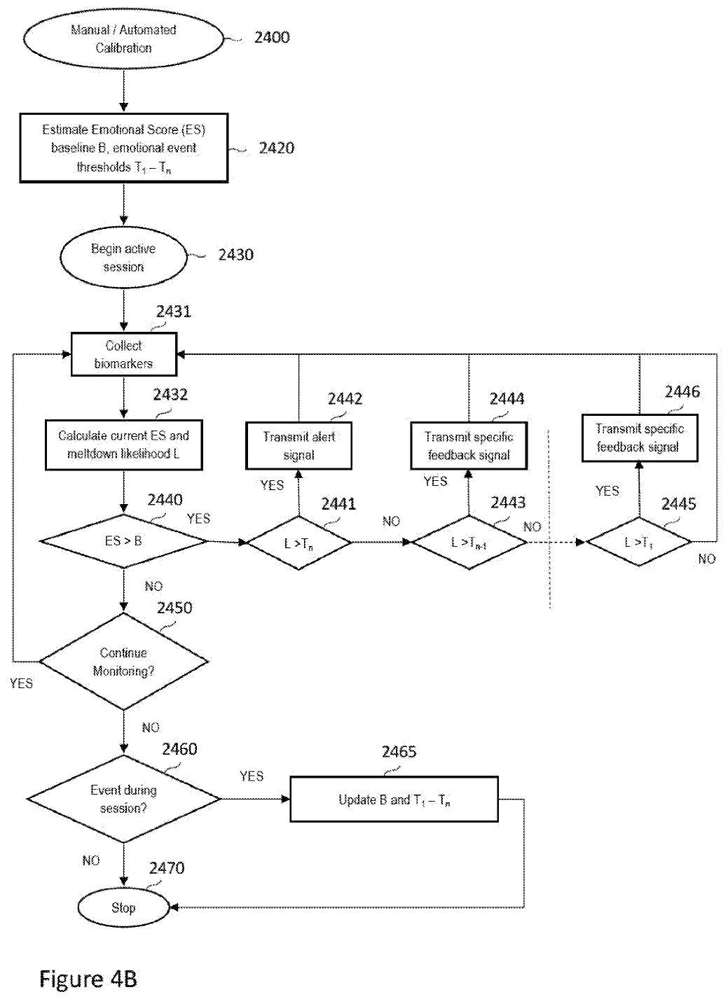

FIG. 4A depicts a flow diagram of a detection and intervention algorithm that may be performed by the control module 22. In response to the actuation of the button (step 400), the device may collect a base state of biometric data (step 410). More specifically, the actuation of the button may cause an activation signal to be sent by the button 141 to the sensors 24, 25. The data collected by the sensors may be transmitted to the control module 22. In one embodiment, the control module 22 utilizes the base state of biometric data to estimate, in real-time, an alert threshold, T₀, and intermediary thresholds, T₁-Tₙ, for launching the alert and various feedback signals (step 420). Subsequently, an active session is also launched in response to the activation signal sent by the button 141 (step 430). During the active session, the sensors may collect additional biometric data (step 431) and the control module 22 may receive the additional biometric data and calculate the emotional score (ES) (step 432).

At step 440, the emotional score may be compared with the initial threshold T₀. If the emotional score is below the initial threshold T₀ (no branch of step 440), the control module 22 may determine whether or not the monitoring should continue (step 450). If the monitoring should continue (yes branch of step 450), the process returns to step 431. If the monitoring should not continue (no branch of step 450; e.g., a stop signal is received from the button 141), the process continues to step 460.

If the emotional score is above the alert threshold (yes branch of step 440), the device 10 determines whether an alert signal has already been transmitted in a specified period of time before the current time (step 441). If no alert signal has been issued (no branch of step 441), an alert signal is transmitted (step 442). If an alert signal was already transmitted in a specified period of time before the current time (yes branch of step 441), the device 10 compares the emotional score with one or more of the previously estimated intermediary thresholds T₁-T₀ (steps 443, 445), and transmits the corresponding feedback signal to the user (steps 444, 446).

A different number of intermediary thresholds T₁-T₀ can be configured depending on the characteristics of the intervention process. Biofeedback signals can be delivered through vibrations and/or light in different increments of time, duration, magnitude or patterns, as desired and as a function of the emotional score level calculated for the user. For example, a short, sharp and abrupt vibration is emitted at step 442 as an alert signal, and subsequently at step 444, a longer, smoother and gentler vibration is emitted to indicate that the emotional score is decreasing. Concurrently, the visual indicators 20 may display a pattern of light (i) with various attributes of the LED (e.g., intensity, color, ON/OFF, etc.) changing based on the emotional score and (ii) to prompt the user to perform a specific action in the intervention process 36.

The intervention process 36 can be tailored to the user based on the user's specific conditions and characteristics. For example, if the intervention is a breathing exercise, a pattern of light can be used to guide the breathing of the user. In other instances, if the intervention is a meditation routine, different vibrations can be used to guide the user through the meditation routine without the need for the user to look at the device 10. In some embodiments, the intervention can be dynamic, for example, gradually increasing or gradually decreasing in intensity as desired. In one embodiment, the intervention process that is directed by the device 10 can be selected by the user via a system (e.g., a phone) that is communicatively coupled to the device 10.

The process of monitoring the emotional score of the user and providing biofeedback based intervention continues at least until the emotional score drops below the alert threshold, T₀. After that, if at step 450, the signal to stop monitoring is received from the button 141, the method proceeds to step 460, in which the control module 22 determines whether an emotional event has occurred during the session. If so (yes branch of step 460), the control module 22 may update the values of the alert threshold, T₀, and intermediary thresholds, T₁-Tₙ, based on information and data derived from the monitoring session. In one embodiment, machine learning can be used to update these thresholds, while in an alternative embodiment, the update may be based on pre-calculated parameters. After the thresholds have been updated or in the case that no event was detected (no branch of step 460), the process may conclude (step 470).

FIG. 4B depicts a flow diagram of another detection and intervention algorithm that may be performed by the control module 22. At step 2400, the device may begin a manual or automated calibration process. During such calibration process, the controller may estimate a baseline, B, of the emotional score, ES, and emotional event thresholds, T₁-Tₙ for launching the alert and various feedback signals (step 2420). The baseline, B, may be a value corresponding to a non-flareup state of the user determined during an initial calibration period and could be personalized to the user wearing the device, or computed for a target group of users (e.g., users with ADHD). The baseline, B, may change over time as the device aggregates more data across more users. During the calibration process, data values that are suspected to correspond to flareup events may be removed so as to not affect the calculation of the baseline, B. Further, the user may be instructed to (i) only perform the calibration when the user believes he or she is in a non-flareup state, or (ii) repeat the calibration process if data suspected to correspond to a flareup event has been identified during the calibration process or if the user believes that the calibration was performed when the user was not in a calm condition. Further, the calibration may take place at the office of a medical professional and/or for children, the calibration may take place in the presence of parents. The emotional event thresholds, T₁-Tₙ may initially be set empirically (step 2420), and then adjusted following the conclusion of an emotional event of the user (step 2465). At step 2430, an active session is also launched after calibration processes have been completed. After the user begins the active session by putting the wearable device on and turning it on, the wearable device begins to collect the biometric data from the sensors (step 2431) and calculates, based on an onboard algorithm, the current emotional score, ES, and the meltdown likelihood, L, which may be a prediction of how close the user is to a meltdown state (e.g., state in which the user is throwing a tantrum, is verbally confrontational, is not capable of following instructions, etc.) in an upcoming time period (i.e., in the next five minutes, in the next ten minutes, etc.).

At step 2440, the current emotional score, ES, may be compared with the baseline emotional score, B. If the emotional score, ES, is below the baseline, B (no branch of step 2440), the control module 22 may determine whether or not the monitoring should continue (step 2450). If the monitoring should continue (yes branch of step 2450), the process returns to step 2431 where additional biomarkers are collected. If the monitoring should not continue (no branch of step 2450; e.g., a stop signal is received from the button 141), the process continues to step 2460.

If the emotional score is above the baseline, B (yes branch of step 2440), the control module 22 determines whether the meltdown likelihood, L, is greater than the n^{th} emotional event threshold, Tₙ. If so (yes branch of step 2441), an alert signal is transmitted (step 2442). If not (no branch of step 2441), the control module 22 determines whether the meltdown likelihood, L, is greater than the n-1^{th} emotional event threshold, Tₙ₋₁. If so (yes branch of step 2443), a specific feedback signal is transmitted (step 2444). If not (no branch of step 2443), the process continues in a similar manner for other thresholds. If the process reaches step 2445, the control module 22 determines whether the meltdown likelihood, L, is greater than the first threshold T₁. If so (yes branch of step 2445), a specific feedback signal is transmitted (step 2446). If not (no branch of step 2445), the process returns to step 2431 where additional biomarkers are collected.

A different number of emotional event thresholds, T₁-Tₙ, can be configured depending on the characteristics of the intervention process. Biofeedback signals can be delivered through vibrations and/or light in different increments of time, duration, magnitude or patterns, as desired and as a function of the emotional score level calculated for the user. For example, a short, sharp and abrupt vibration is emitted at step 1442 as an alert signal, and subsequently at step 1444, a longer, smoother and gentler vibration is emitted to indicate that the emotional score is decreasing. Concurrently, the visual indicators 20 may display a pattern of light (i) with various attributes of the LED (e.g., intensity, color, ON/OFF, etc.) changing based on the emotional score and (ii) to prompt the user to perform a specific action in the intervention process 36.

The process of monitoring the emotional score of the user and providing biofeedback based intervention continues at least until the emotional score drops below the baseline, B. After that, if at step 2450, the signal to stop monitoring is received from the button 141, the method proceeds to step 2460, in which the control module 22 determines whether an emotional event has occurred during the session. If so (yes branch of step 2460), once the monitoring has been completed, the AI algorithm analyzes the data collected, and updates the baseline, B and emotional event thresholds, T₁-Tₙ as needed based on a multidimensional analysis of the recorded biological signals. In one embodiment, machine learning can be used to update these thresholds, while in an alternative embodiment, the update may be based on pre-calculated parameters. After the thresholds have been updated or in the event that no event was detected (no branch of step 2460), the process may conclude (step 2470).

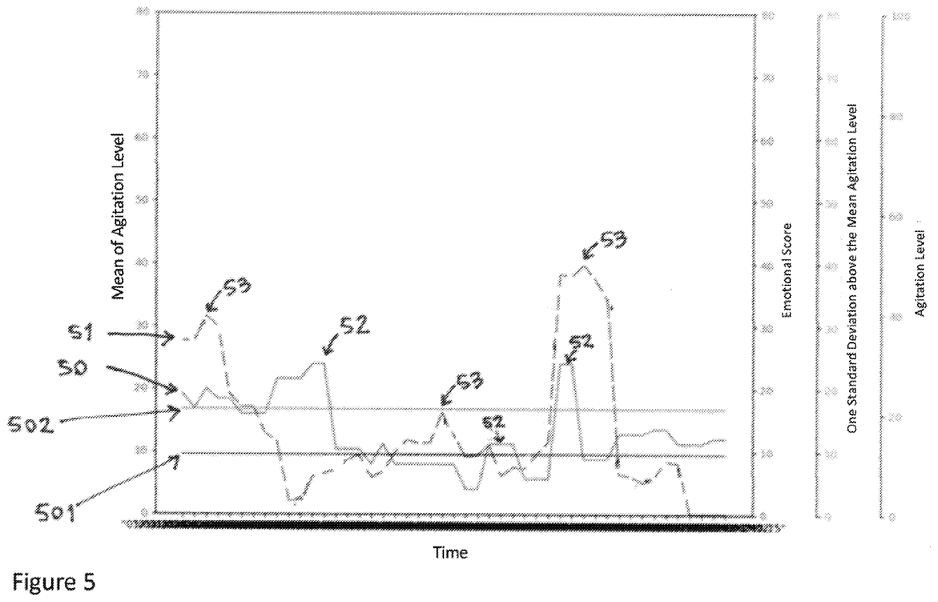

FIG. 5 depicts the emotional score and an agitation level of a user during the same period of time, with both values being displayed on the same plot to allow for easy comparison of these values. In the example depicted in FIG. 5, the data was measured from a user diagnosed with ADHD. The agitation level 50 of the user was observed by external professional observers over time (i.e., the agitation level being one possible external manifestation of emotion). Statistics of the agitation level 50 were computed, for example its mean value (represented as line 501 on the plot), and one standard deviation above the mean value (represented as line 502 on the plot).

The device 10 was worn by the user in order to determine the user's emotional score. More specifically, the emotional score 51 was calculated based on the biomarkers collected by the plurality of sensors of the device 10, using a process of statistical analysis adapted from a previously collected set of data. From the depiction, it can be observed that the peaks 52 in the agitation level 50, which were at or above line 502 (i.e., one standard deviation above the mean), were preceded by peaks of the emotional score 53, indicating that the emotional score 51 could be a valuable predictor for moments of high agitation in the user, and suggesting the usefulness in the monitoring of the emotional score 51 to trigger timely intervention processes to ward off potential flare ups in the user's emotions.

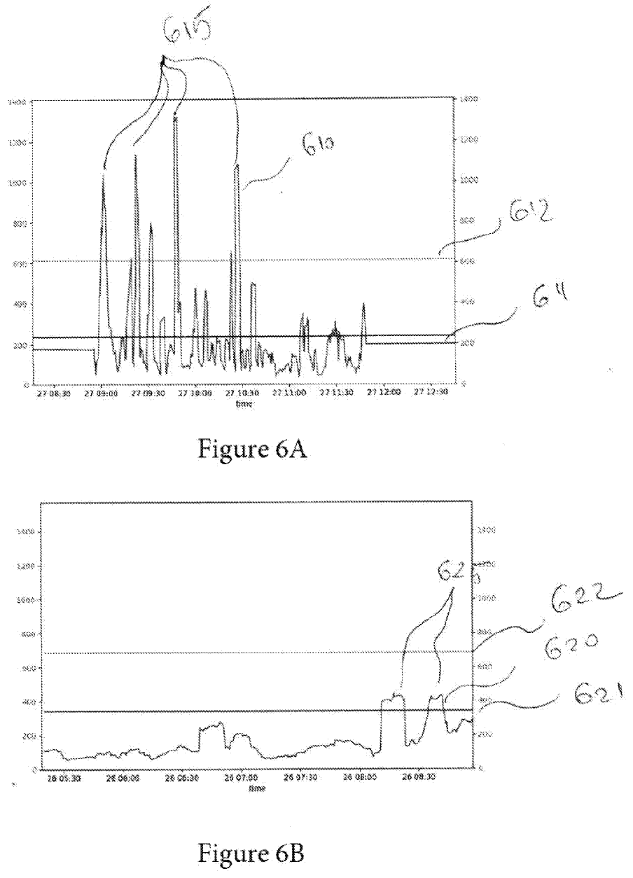

FIGS. 6A and 6B depict the respective emotional scores for two users over time. The data from a first individual diagnosed with ADHD is depicted in FIG. 6A, and the data from a second individual that was not diagnosed with any mental health conditions is depicted in FIG. 6B. Statistics of the emotional score 610 for the first individual were computed during a calibration period (i.e., prior to the active session), including the mean (represented as line 611 in the plot of FIG. 6A) and one standard deviation above the mean value (represented as line 612 in the plot of FIG. 6A). Similarly, statistics of the emotional score 620 for the second individual were computed during a calibration period (i.e., prior to the active session), including the mean (represented as line 621 in the plot of FIG. 6B) and one standard deviation above the mean value (represented as line 622 in the plot of FIG. 6B).

The emotional score 610 of the first individual (diagnosed with AMID) exhibited numerous peaks 615 above line 612 (i.e., one standard deviation above the mean), which was selected as the alert threshold, T₀. In accordance with the algorithm depicted in FIG. 4, an alert signal was transmitted in each case to the individual and an intervention process was launched, leading to an immediate decrease of the emotional score 610, and eventually leading to the emotional score being maintained below the alert threshold, T₀.

The emotional score 620 of the second individual (not diagnosed with any mental health conditions) presented much lower amplitude peaks 625, all of them below line 622 (i.e., one standard deviation above the mean). In order to evaluate the effect of the device 10, the alert threshold, T₀, was manually modified to be the mean of the emotional score during the calibration period. Each time the emotional score exceeded the alert threshold, T₀, an alert signal was transmitted and the intervention process was launched, similarly to the first individual. It can be observed for the second individual that the interventions also caused a decrease in the emotional score, but the decrease occurred over a longer time period.

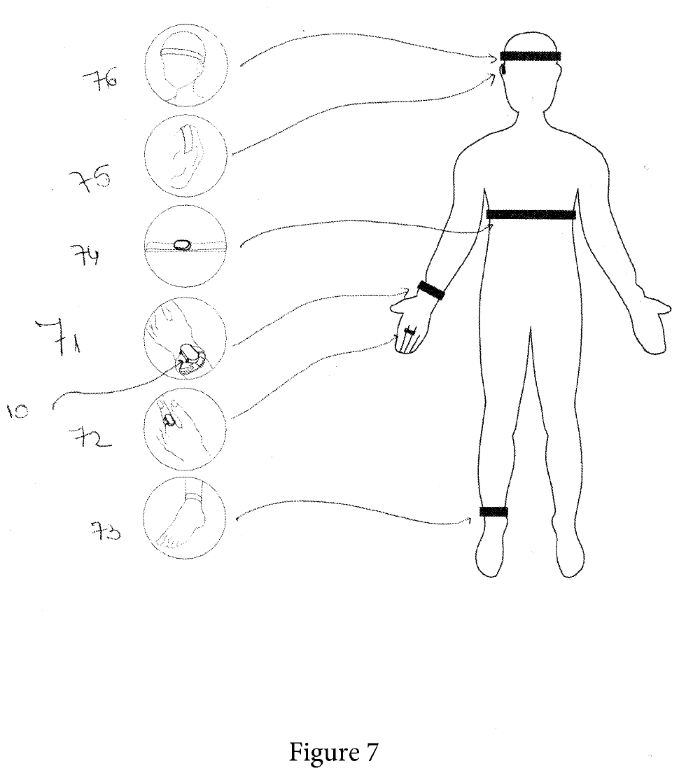

FIG. 7 illustrates several embodiments of the wearable device 10. In a preferred embodiment, the wearable device 10 is integrated into a bracelet or wristband that is worn on the wrist 71. In another embodiment, the wearable device 10 is worn on the ankle 73. In another embodiment, the device 10 is fitted with accurate straps and worn on the chest 74 or on the head 76, in the case that more complex electroencephalogram (EEG)/electrocardiogram (EKG) sensors are employed. In another embodiments, the wearable device 10 is housed within the previously described upper and lower cases 14, 15, allowing the device 10 to be worn as a ring on the finger 72, or as a clip on the ear 75.

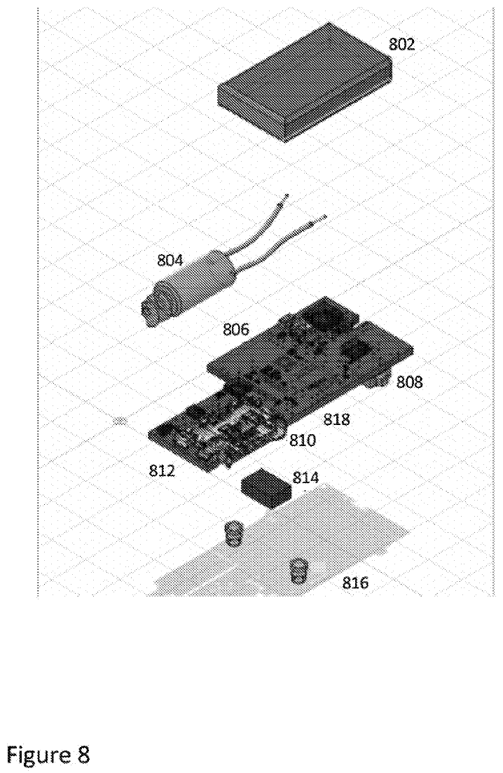

FIG. 8 depicts an illustration of components of the wearable device. The wearable system is defined by a hardware platform that integrates a number of sensors in a space-efficient printed circuit (PC) board and the embedded software that runs on an embedded microcontroller and handles data sampling, processing (including AI/ML methods), communication and haptic feedback. The components include a battery 802, a vibration motor 804, a PC board 806, a multifunction button 808, a USB type C connector 810, a red-green-blue RGB LED 812, a photoptethy srnography (PPG) optical sensor 814, galvanic skin response (GSR) electrodes 816, and a temperature sensor (not visible) and an accelerometer (not visible).

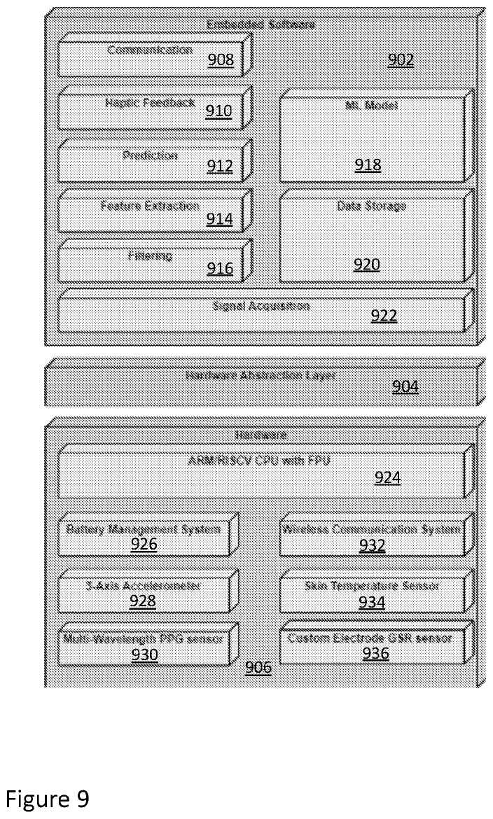

FIG. 9 depicts a hardware and software architecture of the wearable device. A hardware abstraction layer 904 interfaces the embedded software 902 with the hardware 906 of the wearable device. The embedded software 902 may include various software modules, including a communication module 908, a haptic feedback module 910, a prediction module 912, a feature extraction module 914 a filtering module 916, a machine learning (ML) module 918, a data storage module 920 and a signal acquisition module 922. The functionality of various ones of these module will be described hereinbelow.

The hardware 906 may include various devices/components, including an embedded processor 924 (i.e., microcontroller) that has a floating point unit (FPU) unit to help with the digital signal processing tasks, and is low-power to enable longer battery life. The hardware 906 may also include a wireless communication system 932 to communicatively couple the wearable device to an external device via a wireless protocol, such as Bluetooth, Thread, ZigBee and WiFi. The hardware 906 may also include a GSR sensor 936 that is implemented using a low voltage technique utilizing precision operational amplifiers. This method avoids needing to boost the skin electrode voltage to high levels. The hardware 906 may also include an embedded flash memory (not depicted) that allows the wearable device to store large amounts of data without being paired to a smartphone or PC and also provides local storage for the ML model data, enabling AI/ML data processing to be performed on the device itself, independently of an external device such as a smartphone or PC. The hardware 906 may also include a battery management system 926 for managing the usage of the battery 802, a 3-axis accelerometer for sensing movement of the individual wearing the device, a multi-wavelength PPG sensor 930, and a skin temperature sensor 934 for measuring the skin temperature of the individual.

Photoplethysmography (PPG) is a technique employing one or more optical sensors that makes measurements at the surface of the skin to detect volumetric changes in blood circulation. More specifically, PPG uses low-intensity infrared (IR), red and green light. Since light is more strongly absorbed by blood than the surrounding tissues, the changes in blood flow can be detected by PPG sensors as changes in the intensity of light. A voltage signal from a PPG sensor is thus proportional to the volume of blood flowing through the blood vessels. Volumetric changes in blood flow are associated with cardiac activity, hence changes in a PPG signal can be indicative of changes in cardiac activity. The PPG signal itself is typified by a waveform that includes an alternating current (AC) component superimposed on a direct current (DC) component. The AC component corresponds to variations in blood volume in synchronization with a heartbeat, while the DC component is determined by tissue structures surrounding the blood vessels where measurements are taken and other factors and may vary with respiration. By analyzing the PPG signal, various physiological biomarkers may be extracted, including blood oxygen saturation, blood pressure, heart rate, heart rate variability, and other cardiovascular parameters. The user's emotional score is derived as a function of the various measured parameters obtained through analysis of the PPG signal, and, optionally, additional parameters that may be measured using other sensors. In one embodiment, the radial basis function kernel is used to compute the emotional score from the various measured parameters (also called extracted features).

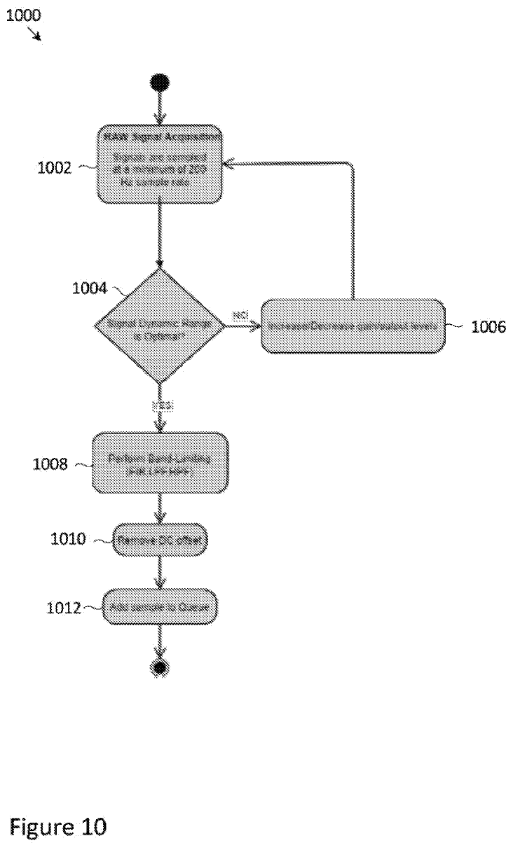

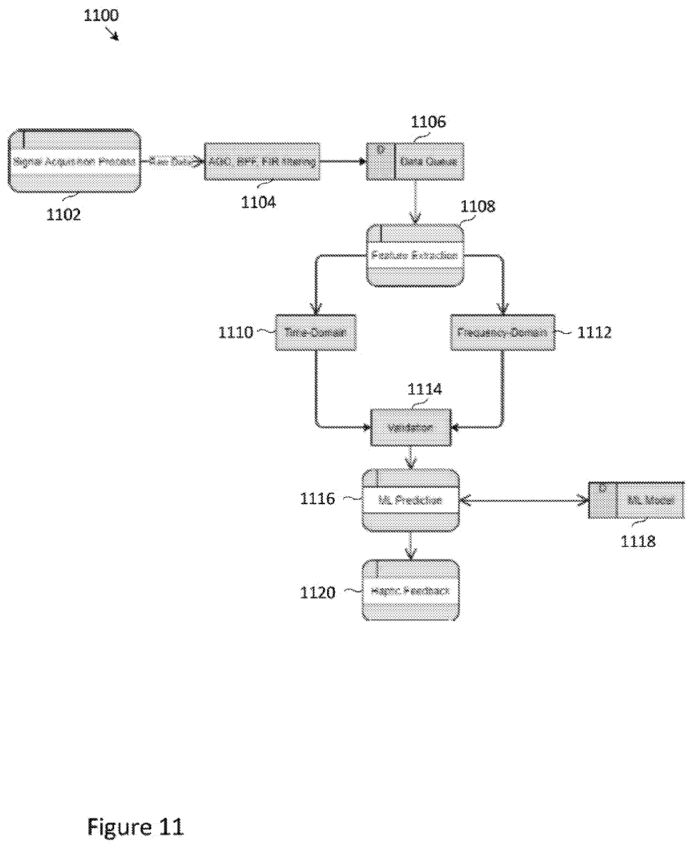

FIG. 10 depicts a flow diagram 1000 of a process for processing the PPG signal. It is noted that such processing may be specific to the PPG signal and may not be needed for the other measured signals (e.g., the body temperature, heart rate, GSR, etc.). At step 1002 (i.e., during the raw signal acquisition phase), the PPG signals (e.g., intensity of IR, RED and GREEN light) from the photoplethysmography (PPG) optical sensor 814 are sampled by the microcontroller 924 with a minimum sampling rate of 200 Hz. At step 1004, the microcontroller 924 determines whether the dynamic range of the signal is optimal (e.g., comparing the dynamic range to a threshold value). If not (no branch of step 1004), an automatic gain control algorithm is used to adjust (e.g., increase or decrease) the output drive level of the signal and the input amplification in order to maximize the dynamic range of the samples (step 1006). Following step 1006, the sampling of the PPG signals may resume (step 1002). If the signal dynamic range is optimal (yes branch of step 1004), the PPG signals are then band-filtered (step 1008) and the steady state DC-offset is removed (1010). Finally, the processed PPG signals may be added to a queue (step 1012) to the feature extraction module (1108), described in FIG. 11.

FIG. 11 depicts a flow diagram 1100 that provides an overview of a process for generating biofeedback to the one or more features extracted from the measured signals. At step 1102, one or more signals may be acquired. At step 1104, automatic gain control (AGC), band pass filtering (BPF), finite impulse response (FIR) filtering and other signal processing operations may be performed on certain ones of the acquired signals, while omitted for other ones of the acquired signals. At step 1106, the processed one or more signals may be added to a queue (as previously described in step 1012). At step 1108, relevant biomarker features may be extracted in the time domain generating time-domain features 1110, and extracted in the frequency domain generating frequency-domain features 1112. The relevant biomarker features may include one or more of skin temperature, heart rate, acceleration vectors, the galvanic skin response (GSR), the skin's electrical resistance/conductivity, the autonomic nervous system (ANS) response (including the high frequency (HF) and low frequency (LF) power), the oxygen saturation percentage of the blood (SpO2%), and respiration rate.

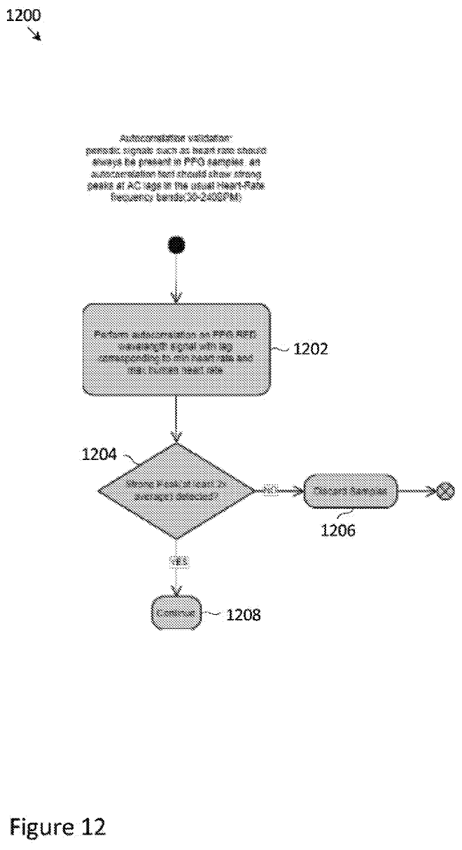

At step 1114, the time-domain features 1110 may be compared to the frequency-domain features 1112 to determine which samples of the signals to discard and which samples of the signals to further analyze. Such “validation” step is further described below in FIG. 12. At step 1116, a machine learning (ML) prediction module makes a prediction as to whether a stressful event has occurred or will soon occur based on the ML model 1118. In another embodiment, the prediction may employ a support vector machine (SVM), in which the dimensions of the SVM may be assigned the above-noted biomarker features. Upon a stressful event being detected or predicted by the ML prediction module (or an SVM), the wearer may be alerted by means of haptic and/or optical feedback (step 1120).

FIG. 12 depicts a flow diagram 1200 of a process for discriminating between samples that should be stored and processed and samples that should be discarded. Sometimes, the signal quality can be severely degraded (e.g., due to improper skin contact, loose strap, etc.), and thus those sample with poor signal quality should be discarded. The quality of the PPG signal may be used as a proxy for the quality of all the measured signals. If the quality of the PPG signal is low, this may indicate that the contact of the device with the skin is not good, and all other signals are deficient and should be discarded during the same time period in which the quality of the PPG signal is low.

The process depicted in FIG. 12 has been developed to determine the quality of the PPG signal within a certain time window based on the PPG red wavelength signal. At step 1202, the microcontroller 924 may auto-correlate the PPG red wavelength signal for a range of lag values, with the minimum lag value corresponding to the minimum heart rate and the maximum lag corresponding to the maximum human heart rate. At step 1204, a determination is made as to whether a strong peak in auto-correlation of the PPG red wavelength signal (e.g., at least 2 times the average) has been detected. If not (no branch of step 1204), the samples (i.e., all samples including the temperature, GSR, etc. within this window) are discarded (step 1206). If so (yes branch of step 1204), the samples (i.e., all samples including the temperature, GSR, etc. within this window) are stored and the process continues to step 1116 in FIG. 11 (step 1208). As a result of the process depicted in FIG. 12, an early validation of the viability of the samples is established, before moving on to the more energy intensive signal processing operations.

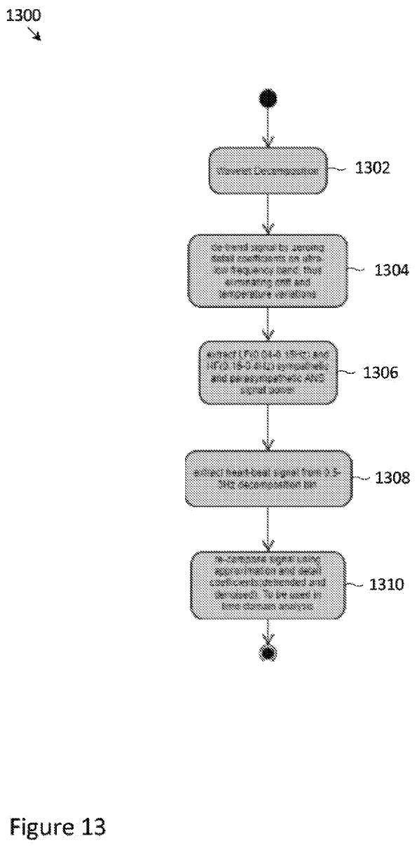

FIG. 13 depicts a flow diagram 1300 of a process to extract features from certain frequency bands from the photoplethysmography (PPG) signals, the process using the wavelet transform, a common type of time-frequency-domain analysis. For instance, one frequency band may correspond to the user's heart rate, one frequency band may correspond to the user's breathing, one frequency band may correspond to the user's ANS response, etc.

At step 1302, the microcontroller 924 may perform a wavelet decomposition of the PPG signals. The wavelet decomposition may utilize a tailor-made wavelet that closely approximates the heart-beat waveform (i.e., a waveform in the time domain that has the systolic and diastolic peaks), and results in very good accuracy in the extraction of features. At step 1304, the PPG signals may be processed in an operation known is “de-trending” in which the detail coefficients on the ultra-low frequency band of the PPG signal are zeroed, thereby eliminating the drift and temperature variations of the PPG signal. At step 1306, the microcontroller 924 may extract the low frequency (i.e., 0.04-0.15 Hz) and high frequency (i.e., 0.16-0.4 Hz) sympathetic and parasympathetic autonomous neural system (ANS) signal power from the PPG signal. At step 1308, the microcontroller 924 may extract a heart-beat signal from the 0.5-3 Hz decomposition bin of the wavelet decomposition. Finally, at step 1310, the microcontroller 924 may compose the decomposed signals to form a much cleaner signal (e.g., without the unwanted components). More specifically, the composition may use the approximation and detail coefficients (e.g., detrended and denoised) that are split using filter banks.

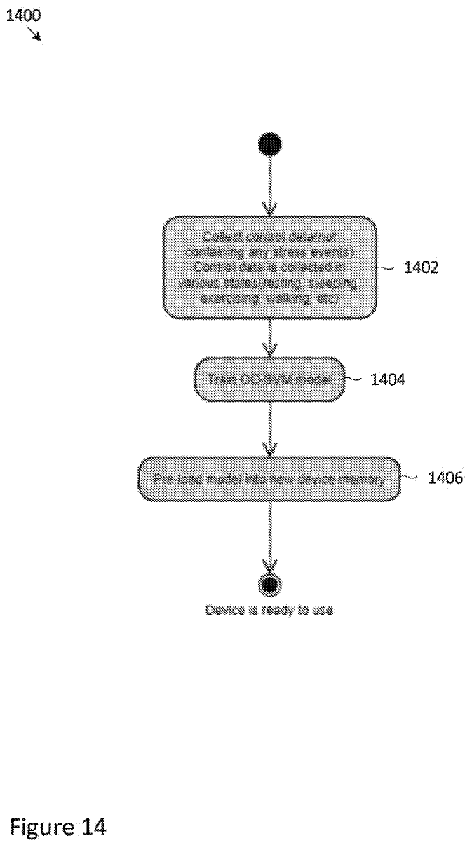

FIG. 14 depicts a flow diagram 1400 of a process for training and initializing the machine learning (ML) model that infers stressful events from the sensor signals. At step 1402, the device collects the control data, which does not include any events that are deemed stressful by the user. The control data is collected in various states (e.g., resting, sleeping, exercising, walking, etc.). At step 1404, the ML model (e.g., a one class support vector machine (OCSVM) model) is trained using the collected control data. Finally, at step 1406, the pretrained ML model is loaded into the memory of a new (blank) device. In summary, at the production stage, blank devices are initialized with a generic ML model trained with data collected from a large number of users, establishing a good baseline for first-use predictions. This generic ML model can also be updated over-the-air when the device is paired with a smartphone/PC.

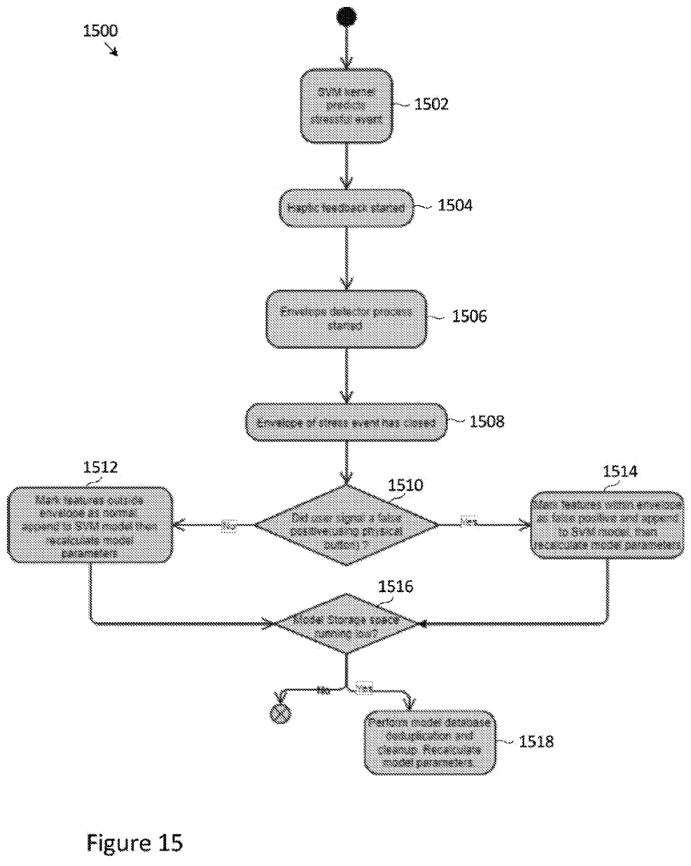

FIG. 15 depicts a flow diagram 1500 of a process for updating the ML model based on feedback from the user received from the physical button 808 of the wearable device. Such process allows the wearable device to predict emotional flares more accurately as the training data is expanded with additional user-specific signals. The process in FIG. 15 utilizes a one class support vector machine (OC-SVM) model in which the one class corresponds to all measurements collected while a stressful event is NOT occurring (from the point of view of the user), such class including events such as the user exercising, eating, talking, etc. under non-stressful conditions.

At step 1502, a support vector machine (SVM) kernel is used to predict stressful events. At step 1504, a haptic feedback is started in response to the prediction of a stressful event. At step 1506, an envelope detector process is started, and indicates the beginning of a stressful event. More specifically, the envelope detector process begins when the output of a radial basis function kernel exceeds a threshold value. At step 1508, the envelope of a stress event is closed, and indicates the conclusion of a stressful event. More specifically, in step 1508, the output of a radial basis function kernel falls below the threshold value.

At step 1510, the microcontroller 924 determines whether the user transmitted a false positive signal (e.g., using the physical button 808 on the device). If so (yes branch of step 1510), the microcontroller 924 marks features extracted within the envelope as a false positive and appends all the extracted features (e.g., heartbeat, GSR, etc.) within the envelop (i.e., within the window of time from which stress event was detected to when stress event ended) to the ML model and then recalculates the model parameters based on the appended data (step 1514). If not (no branch of step 1510), the microcontroller 924 marks features outside of the envelope (i.e., outside the window of time from which stress event was detected to when stress event ended) as normal, and then appends all the extracted features that were recorded before and after the stress event to the ML model and recalculates the model parameters based on the appended data (step 1512). After the completion of either step 1512 or step 1514, the microcontroller 924 determines whether the storage space storing the ML model is running low (step 1516). If not (no branch of step 1516), the process concludes. If so (yes branch of step 1516), the microcontroller 924 performs a model database deduplication and cleanup in order to free up some memory. Further at step 1518, the parameters of the ML model may be recalculated.

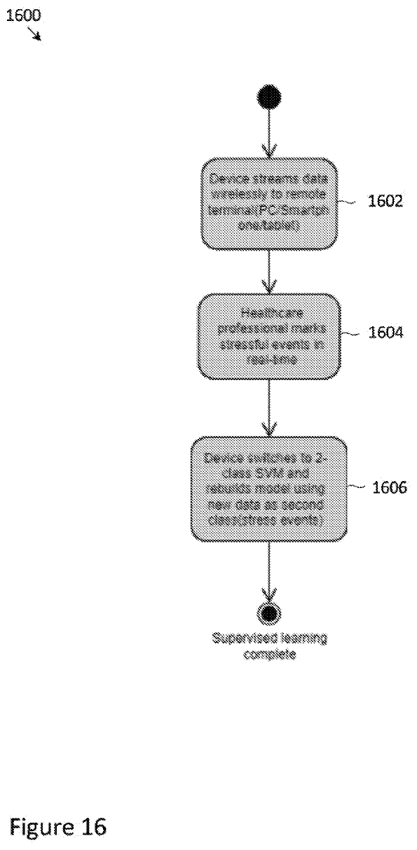

FIG. 16 depicts a flow diagram 1600 of a process for updating the ML model using supervised learning. In a clinical therapy scenario, the wearable device can be even further improved by switching to a fully supervised training mode in which a healthcare professional can observe the user (i.e., wearer of the device) and mark stressful events in real-time (i.e., marks the start time of stress event and end time of stress event), providing the ML classifier with 2-class information. The first class may correspond to all measurements collected while a stressful event is NOT occurring (from the point of view of the user). The second class may refer to all measurements that are collected during a period of time in which a healthcare professional considers a stress event to be happening. At step 1602, the device streams data wirelessly to a remote terminal (e.g., PC, smartphone, tablet, etc.). At step 1604, the healthcare professional marks, in real-time, stressful events experienced by the user. At step 1606, the device switches to a 2-class SVM and rebuilds the ML model using the new data (i.e., events labeled as stressful) as a second class.

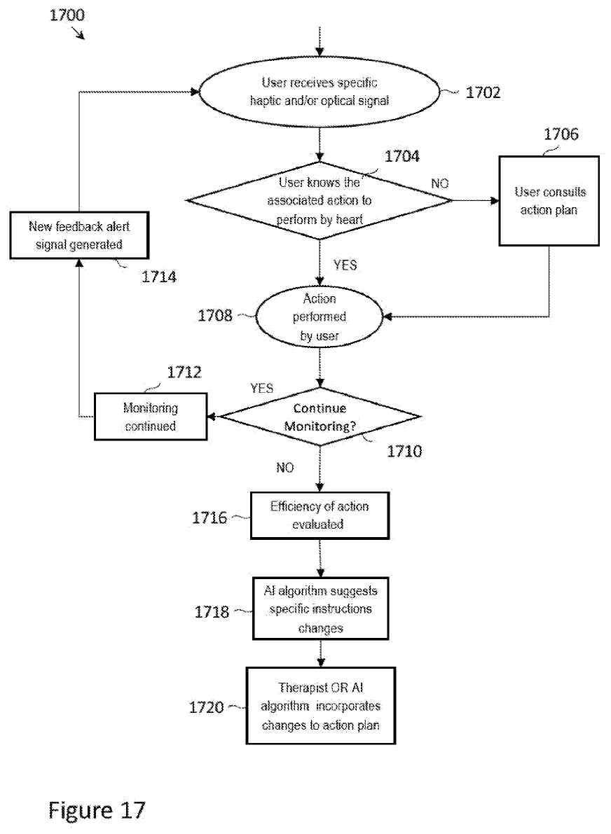

FIG. 17 depicts a flow diagram 1700 of a method of using the alert signals from the high intensity emotional events forecasting process (FIG. 11) to manage the behavioral response of the user to emotional events, to personalize the actions for the user to perform in response to emotional events and to teach the user the appropriate responses to emotional events. The process begins when the user receives a specific alert signal from the wearable (step 1702), in the form of a haptic (vibration) and/or optical signal. The meaning of the alert signal to the user may depend on whether the user is currently being treated by a medical professional (e.g., therapist) and/or whether the user has selected one or more action plans on his/her user profile. If the user is currently being treated by a medical professional, the medical professional may create a personalized plan of action for the user, in which each signal corresponds to a recommended specific action, for example, a breathing exercise, a physical exercise (e.g., stretching or running) or a meditation routine. For example, a red light displayed on the device may instruct the user to perform a breathing exercise, a green light displayed on the device may instruct the user to perform a physical exercise, and a yellow light displayed on the device may instruct the user to perform a meditation routine. Furthermore, the alert signal may also indicate to the user how long the action should be performed (e.g., the indicator stays on for as long as the action should be performed).

The user may be provided with an action plan in hardcopy form from the medical professional that describes each of the alert signals and associates each alert signal with an action to be performed by the user. Alternatively or in addition, the action plan may be provided electronically by the medical professional and may be accessed by the user via a mobile application (e.g., first logging into his/her account and then viewing the instructional material). If the user is not currently being treated by a medical professional, one or more action plans may be provided on the user's account, and the user can select one of the action plans based his/her specific condition/conditions and emotional management goals.

When the user receives an alert signal, if he/she knows the associated action to perform by heart (yes branch of step 1704), the action may be performed by the user (step 1708). If the user does not know the associated action by heart (no branch of step 1704), he/she can consult the action plan (step 1706) to determine and perform the associated action (step 1708). At step 1710, the system determines whether the monitoring of the user should continue (e.g., determine whether the user is still wearing the device). If so (yes branch of step 1710), the monitoring is continued (step 1712), subsequently, a new alert signal is generated (step 1714) and the process is continued from step 1702.

If the monitoring has concluded (no branch of step 1710), the AI algorithm instantiated on the microcontroller 924 may evaluate the efficiency or the effectiveness of the action plan (step 1716) by evaluating an impact of the performed action on the emotional score of the user. Specifically, the AI algorithm may compare the measured characteristics of the emotional event (e.g., length, intensity and evolution of the emotional event; amplitude, slope of emotional score) with preset and/or forecasted models. Such information (including the efficiency or the effectiveness of the action plan, the measured characteristics of the emotional event, the comparison of the emotional event to preset and/or forecasted models) can be transmitted to the medical professional who can use it to further the diagnosis of the user/patient and evaluate the efficiency/effectiveness of the treatment plan (which may include the action plan as well as other treatments such as medication). The AI algorithm can also suggest changes to the action plan (step 1718) based on the effectiveness of the action plan, for example, changes to the action corresponding to an alert signal, changes to the intensity of an alert signal, changes to the duration of an alert signal, etc. These recommendations can either be immediately incorporated into the user's action plan or alternatively can be first transmitted to the medical professional for review prior to being incorporated into the user's action plan (step 1720). In addition, the medical professional can determine changes to the action plan in addition to those suggested by the AI algorithm and request that those changes be incorporated into the action plan. Subsequently, those request changes from the medical professional can be incorporated into the action plan. Through frequent repetition of the actions, the user learns the recommended actions. In time, it is possible for the user to realize the association between various ones of his/her own biological signals (e.g., faster heartbeat or respiration, increased temperature, minute perspiration, etc.) with the actions prescribed by the medical professional or AI algorithm.

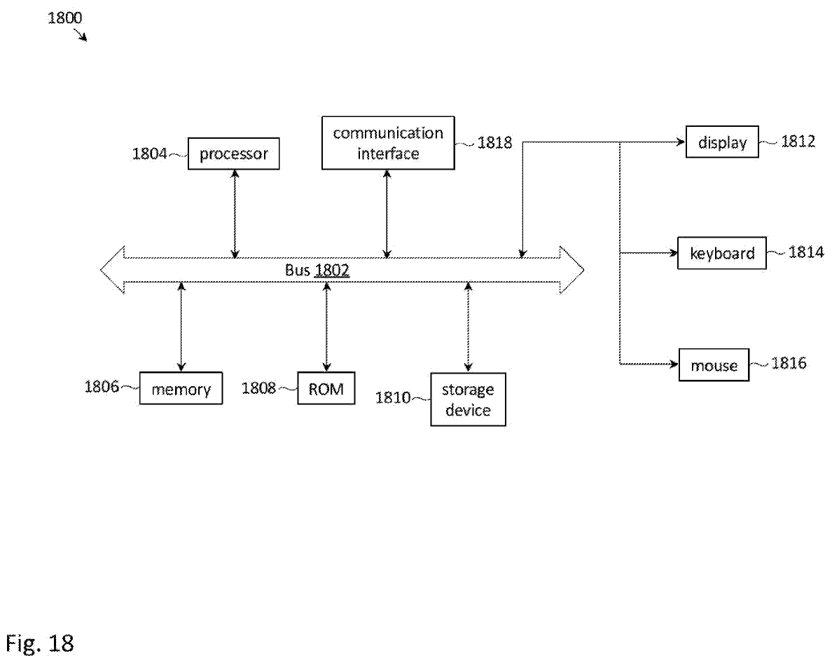

As is apparent from the foregoing discussion, aspects of the present invention involve the use of various computer systems and computer readable storage media having computer-readable instructions stored thereon. FIG. 18 provides an example of a system 1800 that may be representative of any of the computing systems discussed herein. Examples of system 1800 may include a wearable device, a smartphone, a desktop, a laptop, a mainframe computer, an embedded system, a machine, etc. Note, not all of the various computer systems have all of the features of system 1800. For example, certain ones of the computer systems discussed above may not include a display inasmuch as the display function may be provided by a client computer communicatively coupled to the computer system or a display function may be unnecessary. Such details are not critical to the present invention.

Computer system 1800 includes a bus 1802 or other communication mechanism for communicating information, and a processor 1804 coupled with the bus 1802 for processing information. Computer system 1800 also includes a main memory 1806, such as a random access memory (RAM) or other dynamic storage device, coupled to the bus 1802 for storing information and instructions to be executed by processor 1804. Main memory 1806 also may be used for storing temporary variables or other intermediate information during execution of instructions to be executed by processor 1804. Computer system 1800 further includes a read only memory (ROM) 1808 or other static storage device coupled to the bus 1802 for storing static information and instructions for the processor 1804. A storage device 1810, for example a hard disk, flash memory-based storage medium, or other storage medium from which processor 1804 can read, is provided and coupled to the bus 1802 for storing information and instructions (e.g., operating systems, applications programs and the like).

Computer system 1800 may be coupled via the bus 1802 to a display 1812, such as a flat panel display, for displaying information to a computer user. An input device 1814, such as a keyboard including alphanumeric and other keys, may be coupled to the bus 1802 for communicating information and command selections to the processor 1804. Another type of user input device is cursor control device 1816, such as a mouse, a trackpad, or similar input device for communicating direction information and command selections to processor 1804 and for controlling cursor movement on the display 1812. Other user interface devices, such as microphones, speakers, etc. are not shown in detail but may be involved with the receipt of user input and/or presentation of output.

The processes referred to herein may be implemented by processor 1804 executing appropriate sequences of non-transitory computer-readable instructions (or non-transitory machine-readable instructions) contained in main memory 1806. Such instructions may be read into main memory 1806 from another computer-readable medium, such as storage device 1810, and execution of the sequences of instructions contained in the main memory 1806 causes the processor 1804 to perform the associated actions. In alternative embodiments, hard-wired circuitry or firmware-controlled processing units may be used in place of or in combination with processor 1804 and its associated computer software instructions to implement the invention. The computer-readable instructions may be rendered in any computer language.

In general, all of the above process descriptions are meant to encompass any series of logical steps performed in a sequence to accomplish a given purpose, which is the hallmark of any computer-executable application. Unless specifically stated otherwise, it should be appreciated that throughout the description of the present invention, use of terms such as “processing”, “computing”, “calculating”, “determining”, “displaying”, “receiving”, “transmitting” or the like, refer to the action and processes of an appropriately programmed computer system, such as computer system 1800 or similar electronic computing device, that manipulates and transforms data represented as physical (electronic) quantities within its registers and memories into other data similarly represented as physical quantities within its memories or registers or other such information storage, transmission or display devices.

Computer system 1800 also includes a communication interface 1818 coupled to the bus 1802. Communication interface 1818 may provide a two-way data communication channel with a computer network, which provides connectivity to and among the various computer systems discussed above. For example, communication interface 1818 may be a local area network (LAN) card to provide a data communication connection to a compatible LAN, which itself is communicatively coupled to the Internet through one or more Internet service provider networks. The precise details of such communication paths are not critical to the present invention. What is important is that computer system 1800 can send and receive messages and data through the communication interface 1818 and in that way communicate with hosts accessible via the Internet. It is noted that the components of system 1800 may be located in a single device or located in a plurality of physically and/or geographically distributed devices.

Thus, a wearable device and method for stress detection, emotion recognition and emotional management has been described. It is to be understood that the above-description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

1. A computer-implemented method, comprising:
for each of a plurality of time periods:
measuring biological signals of a user, the biological signals including a skin temperature, galvanic skin response (GSR) and photoplethysmography (PPG) signal;
computing an autocorrelation of the PPG signal within the time period;
in response to a maximum of the autocorrelation of the PPG signal exceeding a first threshold value, storing the biological signals including the skin temperature, GSR and PPG signal measured within the time period; and
in response to the maximum of the autocorrelation of the PPG signal not exceeding the first threshold value, not storing the biological signals including the skin temperature, GSR and PPG signal measured within the time window;
monitoring, by a wearable device, an emotional score of the user, the emotional score computed based on the stored biological signals of the user;
detecting an emotional event from the monitored emotional score;
transmitting, by the wearable device, an alert signal to the user in response to the detected emotional event, wherein an action plan associates the alert signal with a corresponding action to be performed by the user;
evaluating an effectiveness of the action plan by evaluating an impact of the action on the emotional score of the user;
determining a first change to the action plan based on the evaluated effectiveness of the action plan; and
incorporating the determined first change into the action plan.

2. The computer-implemented method of claim 1, wherein the biological signals further include one or more of a heart rate, a variability in the heart rate, a blood oxygenation level, a pulse rate, a blood pressure level, a position of the user, and movement of the user, and photoplethysmography (PPG) signals.

3. The computer-implemented method of claim 1, wherein transmitting the alert signal to the user comprises one or more of transmitting an optical signal from light emitting diodes (LED) on the wearable device or transmitting a haptic signal from a vibration actuator of the wearable device.

4. The computer-implemented method of claim 1, wherein the emotional event is characterized by an emotional score exceeding a second threshold value.

5. The computer-implemented method of claim 1, wherein the emotional event is characterized by a meltdown likelihood exceeding a second threshold value.

6. The computer-implemented method of claim 1, wherein the action plan is selected by the user or prescribed by a medical professional of the user.

7. The computer-implemented method of claim 1, wherein the action to be performed by the user includes one or more of a breathing exercise, physical exercise or a mediation routine.

8. The computer-implemented method of claim 1, wherein the evaluation of the impact of the action includes one or more of evaluating a duration, an intensity or an evolution of the emotional event.

9. The computer-implemented method of claim 1, further comprising transmitting the first change to the action plan to a medical professional treating the user.

10. The computer-implemented method of claim 9, wherein the determined first change is incorporated into the action plan upon approval by the medical professional.

11. The computer-implemented method of claim 9, wherein the wearable device is worn on one or more of a wrist, a finger, an ear, a head, an ankle, and a chest of the user.

12. The computer-implemented method of claim 1, further comprising transmitting the evaluated effectiveness of the action plan to a medical professional.

13. The computer-implemented method of claim 1, further comprising:
receiving a second change to the action plan from a medical professional; and
incorporating the second change into the action plan.

14. The computer-implemented method of claim 1, further comprising:
following the alert signal being transmitted to the user, receiving an indication from the user that the detected emotional event was erroneously detected; and
updating a machine learning model in response to the indication from the user that the detected emotional event was erroneously detected, wherein updating the machine learning model comprises marking all features extracted within a window of time from which the detected emotional event started to when the detected emotional event ended as being associated with a false positive.

15. The computer-implemented method of claim 1, wherein the indication from the user that the detected emotional event was erroneously detected is received in response to the user pressing a button on the wearable device.

16. The computer-implemented method of claim 1, wherein the first threshold value is at least two times an average of the PPG signal.

17. The computer-implemented method of claim 1, further comprising:
performing a wavelet decomposition on the PPG signal so as to decompose the PPG signal into a plurality of frequency bins;
extracting a heart rate of the user from a first one of the frequency bins;
extracting a breathing signal of the user from a second one of the frequency bins; and
extracting an autonomous neural system (ANS) response of the user from a third one of the frequency bins.

18. The computer-implemented method of claim 1, wherein transmitting the alert signal to the user comprises transmitting an optical signal from a light emitting diodes (LED) on the wearable device, wherein a wavelength of the optical signal indicates the corresponding action to be performed by the user and a duration of the optical signal indicates a duration of time the user should perform the corresponding action.

19. The computer-implemented method of claim 1, wherein determining the first change to the action plan comprises one or more of determining changes to an intensity of the alert signal, or changes to a duration of the alert signal.

20. The computer-implemented method of claim 1, further comprising:
predicting, by the wearable device and from the monitored emotional score, a future emotional event in which the user is agitated; and
transmitting, by the wearable device, the alert signal to the user in response to the predicted future emotional event in which the user is agitated.
